(19) United States
(12) Patent Application Publication
(10) Pub. No.: US 2010/0028471 A1
(43) Pub. Date: Feb. 4, 2010

(54) COSMETIC USE OF ACTIVE AGENTS THAT STIMULATE MATRIPTASE EXPRESSION

(75) Inventors: Yannick Gerard Maestro, Martigues (FR); Daniel Michel Paul Bergia, Valbonne (FR); Christelle Marie Suzanne Lasserre, Jersey, NJ (US)

Correspondence Address: YOUNG & THOMPSON, 209 Madison Street, Suite 500, Alexandria, VA, 22314, US

(73) Assignee: CHANEL PARFUMS BEAUTE, Neuilly Sur Seine Cedex (FR)

(21) Appl. No.: 12/442,251

(22) PCT Filed: Sep. 18, 2007
(86) PCT No.: PCT/EP07/59835

§ 371 Date: Mar. 20, 2009

Related U.S. Application Data
(60) Provisional application No. 60/850,276, filed on Oct. 10, 2006.
(60) Provisional application No. 60/850,278, filed on Oct. 10, 2006.

(30) Foreign Application Priority Data
Sep. 20, 2006 (FR) 0608245
Sep. 20, 2006 (FR) 0608256

(51) Int. Cl. A61K 8/97; A61Q 19/00

(52) U.S. Cl. 424/774; 424/725; 424/775

(57) ABSTRACT

The present invention relates to a cosmetic process for caring for human skin, intended to moisturize it and/or to protect it against drying out, comprising the topical application to the skin of a composition containing at least one active agent that stimulates the expression of the matriptase MT/SP1. It also relates to the cosmetic use of such an active agent for moisturizing human skin and/or protecting it against drying out. It further relates to the use of such an active agent to manufacture a pharmaceutical composition intended to prevent and/or alleviate skin tautness, stinging and/or itching and/or lip chapping. The active agents are extracts of Ceratonia siliqua, Cananga odorata hook, Cedrelopsis grevei and Cistus ladaniferus L.

The present invention relates to a cosmetic process for caring for human skin, intended to moisturize it and/or to protect it against drying out, comprising the topical application to the skin of a composition containing an active agent that stimulates the expression of the matriptase MT/SP1.

The skin consists mainly of three layers, namely, starting from the most superficial, the epidermis, the dermis and the hypodermis.

The epidermis consists in particular of keratinocytes (predominant), of melanocytes (involved in skin pigmentation) and of Langerhans cells. Its function is to protect the body against the outer environment and to ensure its integrity, in particular to impair the penetration of microorganisms or of chemical substances, and to prevent the evaporation of water contained in the skin.

To do this, the keratinocytes undergo a process of continuous oriented maturation, during which the keratinocytes located in the basal layer of the epidermis form, at the terminal stage of their differentiation, corneocytes which are dead cells that are completely keratinized in the form of horny envelopes consisting of proteins and lipids such as ceramides. During this differentiation process, intercorneocyte epidermal lipids are also formed and then organized in the form of bilayers (lamellae) in the stratum corneum, which contribute, with the abovementioned horny envelopes, to the barrier function of the epidermis.

The barrier function of the epidermis can, however, be disturbed under certain climatic conditions (under the effect of cold and/or of wind, for example), or else under the effect of stress or fatigue, in particular, thus promoting the penetration of allergens, of irritating agents or of microorganisms which thus bring about drying out of the skin that may produce feelings of discomfort such as tautness or redness, and can also impair the radiance of the complexion and the suppleness of the skin.

In order to prevent this phenomenon or correct it, it is known practice to apply to the skin cosmetic compositions containing hygroscopic agents, such as sugars or polyols, intended to take up the water present in the skin and thus impair its evaporation. Use is also conventionally made of fatty substances that make it possible to form an occlusive film on the skin, which contributes to impairing water evaporation. Moreover, these compositions frequently incorporate active agents that act on one or more of the various biological targets involved either in skin regeneration processes, in particular in keratinocyte differentiation, the synthesis of epidermal lipids and corneocyte cohesion, or in the endogenous synthesis of constituents of the natural moisturizing factor (NMF) of the skin, in particular in the synthesis of proteoglycans.

Examples of such active agents are, in particular, α- and β-hydroxy acids, especially lactic acid, glycolic acid and salicylic acid; urea; or aminosulfonic compounds.

However, there still remains the need to propose new cosmetic active agents for more effectively combating drying out of the skin.

In addition, given the ever-increasing search by consumers for natural products containing as few synthetic ingredients as possible, and the increasingly severe regulatory constraints that weigh heavy on compounds derived from the chemical industry, it will be desirable for these cosmetic active agents to be of plant origin.

Now, it is to the applicant's credit to have shown that it is possible to act topically on a new biological target, i.e. the matriptase MT/SP1, for combating drying out of the skin, to develop a screening test for selecting active agents that act on this target and to identify numerous plant extracts corresponding to this test, thus making it possible to meet the abovementioned needs.

The matriptase MT/SP1 (also referred to as ST14 and TAGD-15) is a type II transmembrane protease widely expressed in most cells of epithelial origin, and in particular by keratinocytes. Recent studies on mice have shown that the removal of the matriptase MT/SP1 results in malformations of the stratum corneum, which has a more compact appearance and a less organized stratification, compromising the epidermal barrier function and in the end resulting in the death of the mouse by dehydration. This protease is, moreover, known to be expressed in a large diversity of human tumors of epithelial origin, and also during cicatrization and in skin diseases such as ichthyosis. In the abovementioned study, the mouse is thus used as a model for studying these pathologies (K. List et al., Oncogene, 2002; 21:3765-3779). A more recent study (List et al., J. Cell. Biol., 2003; 163:901-910) has shown that the matriptase MT/SP1 is a key enzyme in epidermal terminal differentiation and that the matriptase-deficient mouse constitutes a good model for studying severe human “harlequin” ichthyosis.

However, to the applicant's knowledge, cosmetic active agents that stimulate the expression of the matriptase MT/SP1 have never yet been disclosed, nor has it been suggested, a fortiori, to use them by topical application on nonpathological human skin.

A subject of the present invention is thus a cosmetic process for caring for human skin, intended to moisturize it and/or to protect it against drying out, comprising the topical application to the skin of a composition containing at least one active agent that stimulates the expression of the matriptase MT/SP1.

A subject of the present invention is also the cosmetic use of an active agent that stimulates the expression of the matriptase MT/SP1, for moisturizing human skin and/or protecting it against drying out.

Another subject of the present invention is the use of at least one active agent that stimulates the expression of the matriptase MT/SP1 to manufacture a pharmaceutical composition intended to prevent and/or alleviate skin tautness, stinging and/or itching and/or lip chapping.

As a preamble, it is specified that the expression “active agent that stimulates the expression of the matriptase MT/SP1” is intended to mean a compound or (in particular in the case of a botanical extract) a mixture of compounds capable of stimulating the expression of the matriptase MT/SP1 compared with an untreated control, determined in particular using the real-time polymerase chain reaction method (RT-PCR) as described in the examples hereinafter.

The active agent that stimulates the expression of the matriptase MT/SP1 can be used in a proportion of from 0.00001% to 10% by weight, preferably in a proportion of from 0.0001% to 5% by weight, and more preferably in a proportion of from 0.001% to 1% by weight, relative to the total weight of the composition.

The active agents that can be used according to the invention are advantageously botanical extracts, i.e. active agents obtained by extraction, using any type of solvent, of any part of a plant, such as the bark, the wood, the roots, the rhizomes, the stems, the leaves, the fruits or the flowers, for example. Examples of such active agents comprise:
extracts of carob pulp or Ceratonia siliqua or extracts (in particular of dried leaves) of Ylang or Cananga odorata Hook., advantageously obtained by alcoholic extraction using a monoalcohol such as ethanol, methanol or isopropanol, optionally mixed with water, and/or a glycol such as propylene glycol, preferably absent any other solvent;
extracts (in particular of bark) of Katafray or Cedrelopsis grevei, such as an essential oil of this plant, advantageously obtained by hydrodistillation; and
extracts (in particular of branches and/or of leaves) of rockrose or Cistus ladaniferus L., advantageously obtained by liquid/liquid extraction of the hydrodistillation water from this plant, after removal of the essential oil, using an apolar organic solvent having a polarity index of less than 1, such as hexane, cyclohexane, heptane and isooctane, preferably absent any other solvent.

In general, the extraction can be carried out on all or part of the plant under consideration, which can be ground or reduced to pieces in the usual manner. In particular, the carob pulp extract that may be used in this invention may be obtained from carob pods which have optionally been dried and preferably been ground. These pods are advantageously deseeded before they are used.

The extraction is generally carried out by immersing or gently stirring the ground material in one or more of the abovementioned solvents at temperatures ranging, for example, from ambient temperature to 100° C., and advantageously from 40 to 80° C., for a period of approximately 30 min to 50 h, for instance from approximately 30 min to 12 h and preferably from 20 to 40 h. The solution is then preferably filtered in order to remove the insoluble plant substances. The solvent is also, where appropriate, removed if it is a volatile solvent such as, for example, ethanol, methanol, hexane or cyclohexane.

In the case where carob pulp is extracted, for instance, the solvent/material ratio can, for example, be between 1:1 and 100:1, and preferably between 10:1 and 50:1. At the end of this extraction step, a carob oleoresin is obtained.

Said oleoresin may then, according to an advantageous aspect, be subjected to a decoloration step, in particular using active charcoal in the presence of a solvent. This decoloration process comprises bringing the extract obtained after the solvent extraction into contact with active charcoal, in an appropriate solvent. The weight of active charcoal added is preferably between 0.5% and 50% of the weight of the extract. One or more solvents chosen from water, C₁-C₄ alcohols such as methanol, ethanol or isopropanol, polar organic solvents such as propylene glycol or dipropylene glycol, or any other usual solvent in the field, will, for example, be used. The volatile solvents can then be eliminated under reduced pressure.

A process of steam hydrodistillation or entrainment (distillation of a mixture of water and of all or part of the plant under consideration) can also be carried out in order to obtain the extract. Regardless of the nature of the organic compounds used, the boiling temperature of the mixture is generally below 100° C. According to this process, a mixture of organic substances and steam are recovered. The temperature of the mixture remains constant until exhaustion of one of the reactants. Next, using a water condenser, condensation of this gas mixture brings about its separation into two liquid phases:
a water-immiscible upper organic phase, referred to as essential oil, containing the majority of the odorant compounds,
a lower aqueous phase, referred to as aromatic water, which contains only very few of said compounds.

Extraction or hydrodistillation are normal practices in the plant extract field, and those skilled in the art are capable of adjusting the reaction parameters thereof, on the basis of their general knowledge. These extraction processes can optionally be completed with other fractionation steps, such as a short-path distillation (or molecular distillation) step, liquid/liquid extraction, supercritical fluid extraction, tangential filtration or else fractionated distillation.

The active agent that stimulates the expression of the matriptase MT/SP1 is used according to the invention for cosmetic purposes, for promoting the formation of a functional barrier and allowing better moisturization of human skin or protecting it against drying out. The invention consequently aims to improve the appearance of normal, nonpathological human skin, as opposed to skin affected by dermatological diseases such as ichthyosis. The inventors have demonstrated that this effect is achieved through an improvement in the barrier function. The process according to the invention can therefore be used to combat the cutaneous signs resulting from a disturbed but nonpathological barrier function, including roughness of the skin, loss of radiance of the complexion or a dull complexion or loss of suppleness of the skin.

The moisturizing effect of the composition used according to the invention can in particular be measured by corneometry, according to usual techniques well known to those skilled in the art.

Preferably, the active agent used according to the invention, or the composition used in the process according to the invention, are applied to nonpathological dry skin. They can advantageously be applied to the skin of the face, neck and, optionally, low neck or, as a variant, to any part of the body.

The composition containing this active agent can be applied in the morning and/or the evening, over the entire face, eneck and, optionally, low neck, or even body.

The composition used according to the invention generally comprises, in addition to the active agent described above, a physiologically acceptable medium, and preferably cosmetically acceptable medium, i.e. a medium which is suitable for use in contact with human skin without any risk of toxicity, of incompatibility, of instability and of allergic response, and in particular which does not cause any feelings of discomfort (redness, tautness, stinging, etc.) unacceptable to the user.

This medium generally contains water and, optionally, other solvents such as ethanol.

The composition used according to the invention can be in any form suitable for topical application to the skin, and in particular in the form of an oil-in-water, water-in-oil or multiple (W/O/W or O/W/O) emulsion, which can optionally be microemulsions or nanoemulsions, or in the form of an aqueous dispersion, a solution, an aqueous gel or a powder. It is preferred for this composition to be in the form of an oil-in-water emulsion.

This composition is preferably used as a product for caring for or cleansing the skin of the face and/or the body, and it can in particular be in the form of a fluid, a gel or a foam, packaged, for example, in a pump-dispenser bottle, an aerosol or a tube, or of a cream packaged, for example, in a jar. As a variant, it can have the form of a makeup product, and in particular of a foundation or of a loose or compact powder.

It can contain various adjuvants, such as at least one compound chosen from:
oils, which can be chosen in particular from: linear or cyclic, volatile or nonvolatile silicone oils, such as polydimethylsiloxanes (dimethicones), polyalkylcyclosiloxanes (cyclomethicones) and polyalkylphenylsiloxanes (phenyl dimethicones); synthetic oils such as fluoro oils, alkyl benzoates and branched hydrocarbons such as polyisobutylene; plant oils, and in particular soybean oil or jojoba oil; and mineral oils such as paraffin oil;
waxes, such as ozokerite, polyethylene wax, beeswax or carnauba wax;
silicone elastomers obtained, in particular, by reaction, in the presence of a catalyst, of a polysiloxane having at least one reactive group (hydrogen or vinyl, in particular) and bearing at least one terminal and/or lateral alkyl (in particular methyl) or phenyl group, with an organosilicone such as an organohydrogenopolysiloxane;
surfactants, preferably emulsifiers, irrespective of whether they are nonionic, anionic, cationic or amphoteric, and in particular fatty acid esters of polyols, such as fatty acid esters of glycerol, fatty acid esters of sorbitan, fatty acid esters of polyethylene glycol and fatty acid esters of sucrose; fatty alcohol ethers of polyethylene glycol; alkylpolyglucosides; polyether-modified polysiloxanes; betaine and derivatives thereof; polyquaterniums; ethoxylated fatty alcohol sulfate salts; sulfosuccinates; sarcosinates; alkyl phosphates and dialkyl phosphates and salts thereof; and fatty acid soaps;
cosurfactants, such as linear fatty alcohols, and in particular cetyl and stearyl alcohols;
thickeners and/or gelling agents, and in particular hydrophilic or amphiphilic, crosslinked or noncrosslinked homopolymers and copolymers of acryloylmethylpropanesulfonic acid (AMPS) and/or of acrylamide and/or of acrylic acid and/or of acrylic acid salts or esters; xanthan gum or guar gum; cellulose-based derivatives; and silicone gums (dimethiconol);
organic screening agents, such as dibenzoylmethane derivatives (including butylmethoxydibenzoylmethane), cinnamic acid derivatives (including ethylhexyl methoxycinnamate), salicylates, para-aminobenzoic acids, β,β′-diphenyl acrylates, benzophenones, benzylidenecamphor derivatives, phenylbenzimidazoles, triazines, phenylbenzotriazoles and anthranilic derivatives;
inorganic screening agents based on mineral oxides in the form of pigments or nanopigments, which are coated or uncoated, and in particular based on titanium dioxide or zinc oxide;
dyes;
preserving agents;
fillers, and in particular powders with a soft-focus effect, which can in particular be chosen from polyamides, silica, talc, mica, fibers (in particular of polyamide or of cellulose);
sequestering agents such as EDTA salts;
fragrances;
and mixtures thereof, without this list being limiting.

Examples of such adjuvants are mentioned in particular in the CTFA dictionary (International Cosmetic Ingredient Dictionary and Handbook published by The Cosmetic, Toiletry and Fragrance Association, 9th edition, 2002), which describes a great diversity, without limitation, of cosmetic and pharmaceutical ingredients normally used in the skin care industry, which are suitable to be used as additional ingredients in the compositions according to the present invention.

The composition used according to the invention can also provide additional benefits, including a soothing or anti-inflammatory activity, a whitening or depigmenting activity, an anti-ageing activity and/or a cleansing activity.

The composition used according to the invention can also comprise active agents other than those that stimulate the expression of the matriptase MT/SP1, and in particular at least one active agent chosen from: keratolytic agents, and in particular α-hydroxy acids such as glycolic acid, lactic acid and citric acid, and esters thereof or salts thereof; β-hydroxy acids, such as salicylic acid and its derivatives; agents that increase keratinocyte differentiation and/or corneification, either directly, or indirectly by stimulating, for example, the production of β-endorphins, such as extracts of Thermus thermophilus or of Theobroma cacao bean shells, water-soluble extracts of corn, peptide extracts of Voandzeia subterranea and niacinamide; epidermal lipids and agents that increase the synthesis of epidermal lipids, either directly, or by stimulating certain β-glucosidases which modulate the deglycosylation of lipid precursors such as glucosylceramide to ceramides, such as phospholipids, ceramides, or lupin protein hydrolysates and dihydrojasmonic acid derivatives; agents stimulating the expression of fructosamine 3-kinase (FN3K) or its related protein (FN3K RP); humectants, such as polyols, and in particular glycerol, glycosaminoglycans such as hyaluronic acid, sugars, their mixtures like the product sold by PENTAPHARM under the trade name Pentavitin® and their alkyl esters, amino acids such as glycine, arginine, histidine, alanine, threonine, lysine, glutamic acid, taurine, proline, serine and their derivatives, pyrrolidonecarboxylic acid (PCA) and its salts, urea and its derivatives, ectoin, glucosamine, creatine, choline, betaine, mineral salts such as chlorine salts, sodium salts, potassium salts, calcium salts, magnesium salts, zinc salts, manganese salts or phosphate salts, and humectant synthetic polymers such as homopolymers and copolymers of methacryloyloxyethylphosphorylcholine and homopolymers and copolymers of glyceryl (meth)acrylate; filling systems; agents for facilitating percutaneous absorption, such as alcohols, fatty alcohols and fatty acids and their ester or ether derivatives, pyrrolidones, terpenes, essential oils and α-hydroxy acids; antioxidants and/or free-radical scavengers and/or antipollution agents, such as tocopherol and its esters, ascorbic acid and its alkyl and phosphoryl esters and certain extracts of plants or of algae, and in particular of Thermus thermophilus; and mixtures thereof, without this list being limiting.

The abovementioned agents can be optionally vectorized in targeting systems such as liposomes, micelles such as the micelles based on sodium lactate and sodium PCA sold by Laboratoires Sérobiologiques under the trade name Micelles Sèches LS8695, oleosomes, nanocapsules or nanoparticles, or else can be transported in polymeric matrices such as the serine-transporting, film-forming matrix based on acacia gum and alginate available from the company Coletica under the trade name Micropatch®.

The term “filling systems” is intended to mean systems for the cutaneous delivery of compounds capable of absorbing the water contained in the skin and of increasing volume subsequent to this absorption. Examples of such systems are in particular glycosaminoglycan-based filling spheres such as the hyaluronic acid-based or chondroitin sulfate-based spheres sold in particular by the company Coletica.

The cosmetic composition used according to this invention may more specifically contain at least one active agent that stimulates the expression of the matriptase MT/SP1 and at least one active agent chosen from: keratolytic agents, agents that increase keratinocyte differentiation and/or corneification, epidermal lipids and agents that increase the synthesis of epidermal lipids, agents stimulating the expression of fructosamine 3-kinase (FN3K) or its related protein (FN3K RP), humectants, filling systems, and mixtures thereof.

More particularly, this composition may contain, besides the active agent(s) that stimulate(s) the expression of the matriptase MT/SP1, at least one active agent chosen from: an ether of polyalkylene glycol and glycerin, made for instance from polyethylene glycol, polypropylene glycol and/or polybutylene glycol and preferably from a mixture thereof, such as the product sold by NOF under the trade name Wilbride® S-753; a fermented extract of Thermus thermophilus; sodium hyaluronate; and mixtures thereof.

It appeared to the applicant that the combination of the active agents that stimulate the expression of matriptase with one or more of the agents described above made it possible to advantageously combine, in the same formula, the respectively long-term and immediate effects of these two types of active agents and thus to obtain maximum and long-lasting moisturization of the skin.

The invention will now be illustrated by means of the following nonlimiting examples.

### EXAMPLES

### Example 1

### Preparation of an Extract of Carob Pulp

An extract of carob pulp was prepared according to the following steps:

1—92° ethanol extraction

250 kg of dried carob pods from which the seeds had been removed are ground. The ground material is loaded into a 2000-liter continuous reactor. The extraction solvent, consisting of 92° ethanol, is loaded into the reactor and the mixture is heated to 60° C. 7500 liters of solvent are circulated in the reactor for 30 hours, i.e. a solvent/material ratio of 30/1. The solvent is then evaporated off under vacuum.

125 kg of carob cleoresin are obtained. The yield from this step is 50%.

213 Decoloration of the Oleoresin

Two hot washes of the oleoresin are carried out with 96.2° ethanol and active charcoal:

1st wash: 1000 g of oleoresin are mixed with 5000 ml of 96.2° ethanol and 125 g of active charcoal. The mixture is stirred vigorously for 2 hours at 50-60° C. and then left to stand at ambient temperature for 2 hours. After filtration of the solution through a Buchner funnel, the primary filtrate is recovered.

2nd wash: The entire primary filtrate is taken up. 500 ml of 96.2° ethanol and 125 g of active charcoal are added. The mixture is stirred for 2 hours at 50-60° C. and then left to stand at ambient temperature for 12 hours. After filtration of the solution through a Büchner funnel, the final filtrate is recovered.

This filtrate is then filtered again through a conical filter in order to remove the last residues of active charcoal, and then the ethanol is evaporated off using a rotary evaporator under vacuum.

The yield from this decoloration process is 60%.

The total yield from the process is 30%.

### Example 2

### Test for Stimulation of MT/SP1 Matriptase mRNA Expression (RT-PCR)

Extracts Tested:

The activity of three botanical extracts was evaluated, namely:
the extract obtained in Example 1 then diluted to 70 wt % in propylene glycol,
an ethanolic extract of dried leaves of Cananga odorata Hook.,
an essential oil of Cedrelopsis grevei, obtained by conventional hydrodistillation, and
an extract of branches and of leaves of Cistus ladaniferus L., obtained by liquid/liquid extraction of the hydrodistillation water from this plant, after removal of the essential oil (Cistus Water Concentrate® F0940 provided by Biolandes).

Protocol:

The effect of the botanical extracts on the expression of the MT/SP1 matriptase mRNA was evaluated by real-time polymerase chain reaction (RT-PCR), for the purpose of quantifying the expression of the MT/SP1 matriptase messenger RNA in a treated sample compared with an untreated sample. The results are normalized relative to the expression of housekeeping genes in these samples.

The results are expressed as number of times increase or decrease in expression of the target gene (MT/SP1 matriptase) in the treated sample, and not as absolute number of copies.

The sequences of the cDNAs/mRNAs of the genes investigated were obtained from GenBank.

Target gene: MT/SP1 matriptase

Housekeeping gene: β2-microglobulin

All the PCR primers were obtained using the PRIMER3 software from the Whitehead Institute for Biomedical Research.

The mRNA was isolated using the Qiagen RNeasy kit (Qiagen) according to the manufacturer's recommendations. The reverse transcription to cDNA was carried out using the gene Amp RNA PCR kit (Applied Biosystems) according to the manufacturer's recommendations.

The real-time PCR measurement was carried out using the iCYCLER IQ machine (Biorad) with SYBR Green I detection. In all the assays, the cDNA was amplified using a standardized program. Each sample was loaded with supermix TQ SYBR Green I, water and primer (stock); the final amount of cDNA per reaction corresponded to 50 ng of the total RNA used for the reverse transcription.

The relative quantification of the expression of the target gene was carried out using the Pfaffl mathematical model (Pfaffl, M W, Nucleic Acids Res. 29(9), p. E45, 2001).

The test was carried out on normal human keratinocytes in culture, in triplicate, treated with the extracts for six hours. The positive results were confirmed using cells from two different donors.

Results:

The results are reported in Table 1 below:

TABLE 1
Stimulation of the MT/SP1 matriptase mRNA expression

| Active agent | | Matriptase | Standard |
|---|---|---|---|
| tested | Concentration⁽¹⁾ | stimulation* | deviation |
| Ceratonia | 0.1% | +86% | 0.05 |
| siliqua | | | |
| Cananga | 100 ug/ml | +36% | 0.15 |
| odorata | | | |
| Hook.⁽²⁾ | | | |
| Cedrelopsis | 50 ug/ml | +50% | 0.17 |
| grevei | | | |
| Cistus | 0.005% | +55% | 0.09 |
| ladaniferus L. | | | |

⁽¹⁾the concentrations of the extracts are expressed as weight of crude extract per weight or volume of preparation
⁽²⁾extract diluted to 80% by weight in propylene glycol
*relative to the untreated control

It emerges from this test that the active agents tested make it possible to stimulate the activity of the matriptase MT/SP1. Although this effect is small in size, it was observed in a reproducible manner on the two batches of keratinocytes tested and for various concentrations of extract and in a dose-dependent manner.

### Example 3

### Test for Stimulation of MT/SP1 Matriptase Protein Expression (Immunofluorescence)

Protocol:

The stimulation of matriptase expression obtained with the extracts described in Example 2 was evaluated in a reconstructed skin model.

This model was prepared in the following way: a solution of collagen containing rat tail collagen type I (BD), 10× DMEM medium (Gibco), sodium bicarbonate (Gibco) and fibroblasts was poured into 24 mm cell culture inserts (Falcon, Becton Dickinson, Schwechat, Austria), which were placed in six-well plates (Falcon). After two hours at 37° C., the gels were equilibrated in KGM at 37° C. in an environment containing 5% CO₂/95% air, in a humidified incubator. After two hours, KGM containing keratinocytes was added to the gel. After immersion of the culture overnight, the medium was replaced with serum-free keratinocyte medium (SKDM, which is a Ca²⁺-rich medium consisting of bovine pituitary extract-free KGM, transferrin from Sigma, BSA from Sigma and L-ascorbic acid from Sigma) outside the insert, and the keratinocytes were maintained at the air-liquid interface. The reconstructed skin culture medium was replaced every two days with preheated fresh SKDM, and the culture was pursued for seven days, with or without the active agents at various concentrations.

The reconstructed skins were then prepared for the purpose of analyzing them by immunofluorescence. Sections 5 μm thick were prepared from reconstructed skins fixed with paraformaldehyde and then frozen. The nonspecific binding on the sections was blocked with serum (bovine serum albumin). The reconstructed skin samples thus prepared were incubated with an anti-matriptase antibody (Bethyl Laboratories, TX), and then labeled, in a second step, with a fluorescent-agent-complexed second antibody (anti rabbit Alexa Fluor 546, Molecular Probes, UK). The detection was carried out by immunofluorescence. The slides were examined using a Leica microscope.

Results:

It was observed that the extracts of Ceratonia siliqua (0.1%), Cistus ladaniferus L. (0.005%), Cedrelopsis grevei (50 μg/ml) and Cananga odorata Hook. (100 μg/ml) all stimulated the expression of the matriptase MT/SP1, visible in the stratum granulosum. These results were confirmed using cells derived from two donors.

### Example 4

### In Vivo Experiment

The hydrating effect of an active agent stimulating matriptase expression was evaluated using the following test protocol.

A serum and a gelled fluid were prepared, respectively in the form of a water-in-silicon emulsion and of an oil-in-water emulsion. Each of them contained 0.1 wt % of the carob pulp extract of Example 1.

Three test areas were then drawn on the forearms of 20 female volunteers, each of which formed a 5 cm side square. The first and second areas were respectively coated with 2 mg/cm² of the serum and the fluid. The third area was used as a control.

The hydration obtained at several periods of time has been measured by corneometry and the results have been expressed as an increase in the hydration percentage relative to the control. The mean value of the results thus obtained is indicated in the following Table 2.

TABLE 2
In vivo hydratation

Hydratation

| | | Serum | Fluid |
|---|---|---|---|
| T10 | min | +82.2% | +68.3% |
| T2 | h | +76.4% | +45.7% |
| T4 | h | +73.3% | +37.0% |
| T8 | h | +65.8% | +32.9% |
| T24 | h | +24.3% | +14.5% |

One can derive from this table that the extract that stimulates matriptase expression according to this invention enhances significantly the hydration of skin up to 24 hours after application thereon.

### Example 5

### Cosmetic Compositions

The following compositions can be prepared in a manner conventional to those skilled in the art. The amounts indicated below are expressed as percentages by weight. The ingredients in uppercase letters are identified in accordance with the INCI name.

5A - Cream gel (oil-in-water emulsion)

| | |
|---|---|
| Tetrasodium EDTA | 0.05% |
| Glycerol | 5.00% |
| Aqueous phase gelling agents | 4.00% |
| Alcohol | 3.00% |
| Preservatives agents | 0.50% |
| Extract of Cananga odorata⁽¹⁾ | 0.10% |
| Sodium hyaluronate | 3.00% |
| Cyclomethicone | 8.00% |
| Dimethicone | 3.00% |
| Isononyl isononanoate | 3.00% |
| Fragrance | qs |
| Dyes | qs |
| Water | qs 100.00% |

⁽¹⁾as described in Example 1 and then diluted to 80% by weight in propylene glycol

This composition can be applied daily, in the morning and/or evening, to the facial skin in order to moisturize it and to make it supple, smooth and bright.

5B - Serum (water-in-silicone emulsion)

| | |
|---|---|
| GLYCERYL POLYMETHACRYLATE & PROPYLENE | 10.00% |
| GLYCOL⁽²⁾ | |
| Glycerol | 5.00% |
| Preservatives agents | qs |
| Alcohol | 10.00% |
| Extract of Cedrelopsis grevei⁽³⁾ | 0.50% |
| Mixture of sugars and amino acids⁽⁴⁾ | 3.00% |
| Sodium pyrrolidone carboxylate | 4.00% |
| Sodium hyaluronate | 2.50% |
| SACCHARIDE ISOMERATE⁽⁵⁾ | 1.00% |
| Moisturizing Micelles sèches⁽⁶⁾ | 20.00% |
| Extract of Thermus thermophilus⁽⁷⁾ | 3.00% |
| CYCLOPENTASILOXANE & PEG/PPG-18/8 | 20.00% |
| DIMETHICONE | |
| Dyes | qs |
| Water | qs 100.00% |

⁽²⁾LUBRAJEL MS ® from Guardian Laboratories
⁽³⁾as described in Example 1
⁽⁴⁾HYDRATYL LS 8453 ® from Laboratoires Sérobiologiques
⁽⁵⁾PENTAVITIN ® from Pentapharm
⁽⁶⁾Micelles Sèches LS8695 from Laboratoires Sérobiologiques
⁽⁷⁾Venuceane ® from Sederma

This composition can be applied daily, in the morning and/or evening, to skin that is particularly dehydrated and/or exposed to environmental stress, in order to improve the comfort and the appearance thereof.

5C - Cream (oil-in-water emulsion)

| | |
|---|---|
| Panthenol | 0.40% |
| GLYCERYL POLYMETHACRYLATE & PROPYLENE | 10.00% |
| GLYCOL⁽²⁾ | |
| Carboxyvinyl polymer | 0.60% |
| 25% Sodium hydroxide solution | 0.30% |
| Sodium hyaluronate | 0.30% |
| Glycerol | 3.00% |
| Extract of Cistus ladaniferus L.⁽⁷⁾ | 1.00% |
| Glyceryl stearate | 1.50% |
| Cetyl alcohol | 1.50% |
| Polyoxyethylene stearate (40 EO) | 2.00% |
| Oxyethylenated stearyl alcohol (2 EO) | 0.50% |
| Octyldodecyl neopentanoate | 5.00% |
| C₁₂-C₁₅ alkyl benzoate | 3.00% |
| Plant oils | 3.00% |
| Phytosterol esters | 1.00% |
| Tocopheryl acetate | 1.00% |
| Silicone oils | 4.00% |
| Disodium EDTA | 0.05% |
| Preservatives agents | 0.90% |
| Dyes | qs |
| Water | qs 100.00% |

⁽²⁾LUBRAJEL MS ® from Guardian Laboratories
⁽⁷⁾as described in Example 1

This cream can be applied to dry skin in the morning and/or in the evening, in order to improve the softness and suppleness thereof and to prevent the formation of dehydration lines.

5D - Serum (water-in-silicone emulsion)

| | |
|---|---|
| GLYCERYL POLYMETHACRYLATE & PROPYLENE | 10.00% |
| GLYCOL⁽¹⁾ | |
| Polyols | 5.00% |
| Preservatives agents | Qs |
| Alcohol | 10.00% |
| Extract of Ceratonia siliqua⁽²⁾ | 0.50% |
| PEG/PPG/POLYBUTYLENE GLYCOL-8/5/3 | 2.00% |
| GLYCERIN⁽³⁾ | |
| Sodium pyrrolidone carboxylate | 4.00% |
| Sodium hyaluronate | 2.50% |
| Shea butter | 0.50% |
| Filling spheres based on | 1.00% |
| hyaluronic acid⁽⁴⁾ | |
| Moisturizing Micelles sèches⁽⁵⁾ | 20.00% |
| Extract of Thermus thermophillus⁽⁶⁾ | 3.00% |
| CYCLOPENTASILOXANE & PEG/PPG-18/8 | 20.00% |
| DIMETHICONE | |
| Cyclopentasiloxane | 5.00% |
| Dyes | qs |
| Water | qs 100.00% |

⁽¹⁾LUBRAJEL MS ® from Guardian Laboratories
⁽²⁾as described in Example 1 then diluted to 70 wt % in propylene glycol
⁽³⁾WILBRIDE S-753 from ROSSOW
⁽⁴⁾CB0A068A from COLETICA
⁽⁵⁾Micelles Sèches LS8695 from Laboratoires Sérobiologiques
⁽⁶⁾Venuceane ® from Sederma

This composition may be applied daily onto dehydrated skin to improve its comfort and appearance.

1-21. (canceled)

22. cosmetic process for caring for human skin, intended to moisturize it and/or to protect it against drying out, comprising the topical application to the skin of a composition containing at least one active agent that stimulates the expression of the matriptase MT/SP1.

23. The process as claimed in claim 22, wherein the active agent that stimulates the expression of the matriptase MT/SP1 is a botanical extract.

24. The process as claimed in claim 23, wherein the active agent is an extract of carob pulp (Ceratonia siliqua).

25. The process as claimed in claim 24, wherein the extract can be obtained by alcoholic extraction using a monoalcohol optionally mixed with water and/or a glycol.

26. The process as claimed in claim 24, wherein the extract is obtained from deseeded carob pods.

27. The process as claimed in claim 23, wherein the active agent is an extract of Cananga odorata Hook.

28. The process as claimed in claim 27, wherein the active agent is an extract of dried leaves.

29. The process as claimed in claim 27, wherein the extract can be obtained by alcoholic extraction using a monoalcohol optionally mixed with water and/or a glycol.

30. The process as claimed in claim 23, wherein the active agent is an extract of Cedrelopsis grevei.

31. The process as claimed in claim 30, wherein the active agent is an extract of bark.

32. The process as claimed in claim 30, wherein the extract is an essential oil that can be obtained by hydrodistillation.

33. The process as claimed in claim 23, wherein the active agent is an extract of Cistus ladaniferus L.

34. The process as claimed in claim 33, wherein the active agent is an extract of branches and/or of leaves.

35. The process as claimed in claim 33, wherein the extract can be obtained by liquid/liquid extraction of the hydrodistillation water, after removal of the essential oil, using an apolar organic solvent having a polarity index of less than 1.

36. The process as claimed in claim 22, which is used to combat the cutaneous signs resulting from a nonpathological disturbed barrier function.

37. The process as claimed in claim 36, wherein said signs are chosen from: roughness of the skin, loss of radiance of the complexion and loss of suppleness of the skin.

38. The process as claimed in claim 22, which is carried out on nonpathological dry skin.

39. The process as claimed in claim 22, wherein the composition further contains at least one active agent chosen from: keratolytic agents, agents that increase keratinocyte differentiation and/or corneification, epidermal lipids and agents that increase the synthesis of epidermal lipids, humectants, agents for facilitating percutaneous absorption, filling systems, and mixtures thereof.

40. The process as claimed in claim 39, wherein the active agent is chosen from: a fermented extract of Thermus thermophilus, sodium hyaluronate, and mixtures thereof.
